(19) United States
(12) Patent Application Publication
(10) Pub. No.: US 2006/0276431 A1
(43) Pub. Date: Dec. 7, 2006

(54) Methods for treating severe acute respiratory syndrome

(76) Inventor: Daniel P. Rossignol, Mahwah, NJ (US)

Correspondence Address: VENABLE LLP, P.O. BOX 34385, WASHINGTON, DC, 20043-9998, US

(21) Appl. No.: 11/331,068

(22) Filed: Jan. 13, 2006

Related U.S. Application Data
(63) Continuation of application No. PCT/US04/22123, filed on Jul. 12, 2004.
(60) Provisional application No. 60/486,444, filed on Jul. 14, 2003.

(51) Int. Cl. A61K 31/739

(52) U.S. Cl. 514/54

(57) ABSTRACT

The invention provides methods for treating severe acute respiratory syndrome (SARS) with lipopolysaccharides.

### RELATED APPLICATION

This application is a continuation of PCT/US2004/022123 filed Jul. 12, 2004, which claims priority to U.S. Application No. 60/486,444 filed Jul. 14, 2003, the disclosures of which are incorporated by reference herein in their entirety.

### FIELD OF THE INVENTION

The invention provides methods for treating severe acute respiratory syndrome with lipopolysaccharides.

### BACKGROUND OF THE INVENTION

Severe acute respiratory syndrome (SARS) is a respiratory illness that has recently been reported in Asia, North America, and Europe. Patients with severe acute respiratory syndrome generally experience one or more symptoms that include a fever greater than 100° F., headaches, malaise, and body aches. Some patients also experience mild respiratory symptoms. After 2 to 7 days, patients may develop a dry cough and have trouble breathing.

Severe acute respiratory syndrome may be spread by person-to-person contact. Most cases of severe acute respiratory syndrome have involved people who cared for or lived with someone with the disease, or had direct contact with infectious material (e.g., respiratory secretions) from a patient. Severe acute respiratory distress syndrome can be spread by touching the skin of people or objects that are contaminated with infectious droplets. It is possible that severe acute respiratory syndrome can be spread more broadly through the air or by other ways that are currently not known.

There is a need in the art for therapeutic treatments for severe acute respiratory syndrome. The invention is directed to this, as well as other, important ends.

### SUMMARY OF THE INVENTION

The invention provides methods for treating severe acute respiratory syndrome by administering to a patient in need thereof a therapeutically effective amount of at least one lipopolysaccharide. The invention also provides methods for treating sepsis caused by severe acute respiratory syndrome by administering to a patient in need thereof a therapeutically effective amount of at least one lipopolysaccharide.

### DETAILED DESCRIPTION OF THE INVENTION

The invention provides methods for treating or preventing severe acute respiratory syndrome by administering to a patient in need thereof a therapeutically effective amount of at least one lipopolysaccharide. In one embodiment, the invention provides methods for treating or preventing sepsis caused by severe acute respiratory syndrome in a patient in need thereof by administering a therapeutically effective amount of at least one lipopolysaccharide.

Exemplary lipopolysaccharides include the compounds described in WO 96/39411 and U.S. Pat. Nos. 5,530,113, 5,681,824, 5,750,664, 5,935,938, 6,184,366 and 6,417,172, the disclosures of which are incorporated by reference herein in their entirety. These compounds are generally represented by Formula (A), pharmaceutically acceptable salts thereof, and/or stereoisomers (including enantiomers and/or diastereomers) thereof: 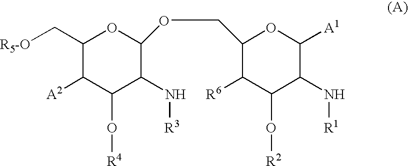
wherein R¹ is selected from the group consisting of:

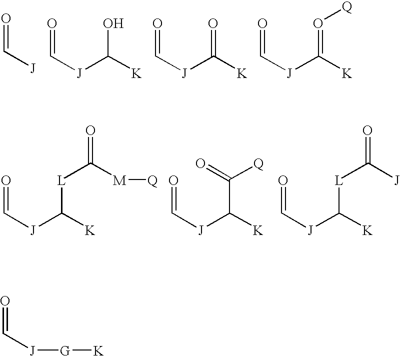

J, K and Q are each independently a straight or branched C₁₋₁₅ alkyl;

L is O, N or C;

M is O or N;

G is N, O, S, SO or SO₂;

R² is a straight or branched C₅₋₁₅ alkyl;

R³ is selected from the group consisting of:

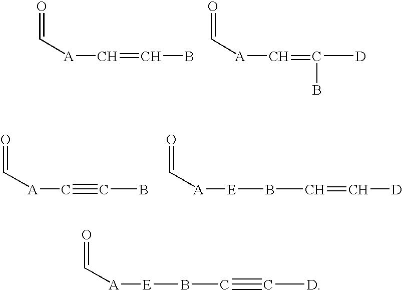

E is N, O, S, SO or SO₂;

A, B and D are each independently a straight or branched C₁₋₁₅ alkyl group;

R⁴ is a straight or branched C₄₋₂₀ alkyl group or

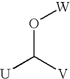

U and V are each independently a straight or branched C₂₋₁₅ alkyl group;

W is a hydrogen or a straight or branched C₁₋₅ alkyl group;

R⁵ is hydrogen, -J′, -J′-OH, -J′-O—K′, -J′-O—K′—OH or J′-O—PO(OH)₂;

J′ and K′ are each independently a straight or branched C₁₋₅ alkyl group;

R⁶ is hydroxy, halogen, a C₁₋₅ alkoxy group or a C₁₋₅ acyloxy group;

A¹ and A² are each independently selected from the group consisting of:

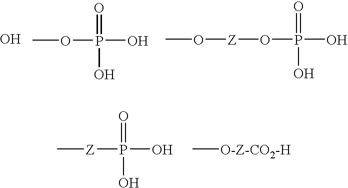

Z is a straight or branched C₁₋₁₀ alkyl group.

The term “alkyl” refers to aliphatic organic groups which may be branched or straight and which may optionally be substituted with one or more halogen atoms at any position along the alkyl chain. The term “pharmaceutically acceptable salt” includes salts of compounds derived from the combination of the compound and an organic or inorganic acid or base.

A preferred compound of Formula (A) is Compound (1), pharmaceutically acceptable salts thereof, and/or stereoisomers (including enantiomers and/or diastereomers) thereof:

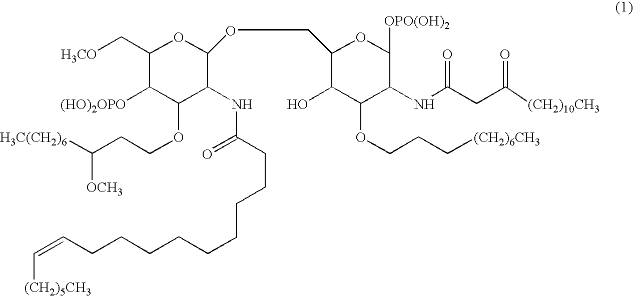

In a preferred embodiment, Compound (1) is Compound (1A) or a pharmaceutically acceptable salt thereof, which is represented by the following formula:

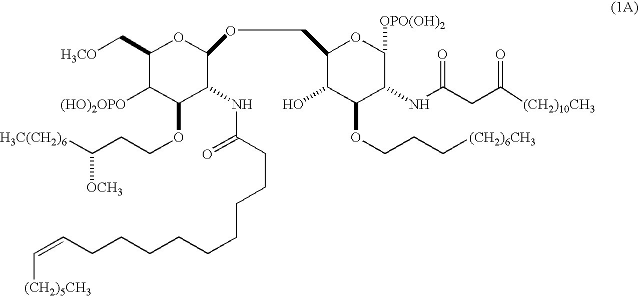

When Compound 1A is a sodium salt (i.e., one or both hydrogen atoms in the —OPO(OH)₂ groups are replaced with sodium), then the compound is E5564.

Other preferred compounds described in WO 96/39411 and U.S. Pat. Nos. 5,530,113, 5,681,824, 5,750,664, 5,935,938, and 6,184,366 for use in the present invention include those of Formula (B), pharmaceutically acceptable salts thereof, and/or stereoisomers (including enantiomers and/or diastereomers) thereof:

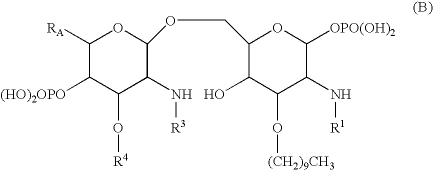

wherein R¹, R³ and R⁴ are as defined below:

| # | R¹ | R³ | R⁴ |
|---|---|---|---|
| 1 | COCH₂CO(CH₂)₁₀CH₃ | CO(CH₂)₉CH═CH(CH₂)₅CH₃ | (CH₂)₂CH(OCH₃)(CH₂)₆CH₃ |
| 2 | COCH₂CO(CH₂)₁₀CH₃ | CO(CH₂)₉CH═CH(CH₂)₅CH₃ | (CH₂)₂CH(OH)(CH₂)₆CH₃ |
| 3 | COCH₂CO(CH₂)₁₀CH₃ | CO(CH₂)₁₆CH₃ | (CH₂)₂CH(OH)(CH₂)₆CH₃ |
| 4 | COCH₂CHOH(CH₂)₁₀CH₃ | CO(CH₂)₉CH═CH(CH₂)₅CH₃ | (CH₂)₂CH(OH)(CH₂)₆CH₃ |
| 5 | COCH₂CO(CH₂)₁₀CH₃ | CO(CH₂)₉CH═CH(CH₂)₅CH₃ | (CH₂)₉CH₃ |
| 6 | CO(CH₂)₉CH═CH(CH₂)₅CH₃ | CO(CH₂)₉CH═CH(CH₂)₅CH₃ | (CH₂)₂CH(OH)(CH₂)₆CH₃ |
| 7 | CO(CH₂)₁₂CH₃ | CO(CH₂)₉CH═CH(CH₂)₅CH₃ | (CH₂)₂CH(OH)(CH₂)₆CH₃ |
| 8 | COCH₂CH(OCH₃)(CH₂)₁₀CH₃ | CO(CH₂)₉CH═CH(CH₂)₅CH₃ | (CH₂)₂CH(OCH₃)(CH₂)₆CH₃ |
| 9 | COCH₂CH(OCH₃)(CH₂)₁₀CH₃ | CO(CH₂)₉CH═CH(CH₂)₅CH₃ | (CH₂)₂CH(OH)(CH₂)₆CH₃ |
| 10 | COCH₂CH(OH)(CH₂)₁₀CH₃ | CO(CH₂)₉CH═CH(CH₂)₅CH₃ | (CH₂)₂CH(OCH₃)(CH₂)₆CH₃ |
| 11 | COCH₂CO(CH₂)₁₀CH₃ | CO(CH₂)₉CH═CH(CH₂)₅CH₃ | (CH₂)₂CH(OCH₃)(CH₂)₆CH₃ |

wherein R_{A} in Compounds (1)-(10) is CH₂OCH₃ and R_{A} in Compound (11) is CH₃.

Methods for making Compounds (1) and (1A) and the compounds of Formulas (A) and (B) are described in WO 96/39411 and U.S. Pat. Nos. 5,530,113, 5,681,824, 5,750,664, 5,935,938, 6,184,366, and 6,417,172, the disclosures of which are incorporated by reference herein in their entirety.

The compounds described herein and in WO 96/39411 and U.S. Pat. Nos. 5,530,113, 5,681,824, 5,750,664, 5,935,938, 6,184,366, and 6,417,172 are administered in dosages which provide a therapeutically effective treatment for severe acute respiratory syndrome; generally, these dosages are between about 0.01 and about 500 mg/patient, between about 0.05 and about 100 mg/patient; between about 1 and about 50 mg/patient; between about 1 and about 25 mg/patient; or between about 1 and about 12 mg/patient. The dosages can be administered over three to six days as a continuous infusion or as an intermittent dosing to obtain desired plasma concentrations. It will be understood, however, that the specific dose level for any particular patient will depend on a variety of factors including the activity of the specific compound used; the age, weight, general health, and sex of the patient being treated; the time and route of administration; the rate of excretion; and other drugs which have previously been administered.

Pharmaceutical compositions containing the active ingredient may be in any form suitable for the intended method of administration. Aqueous solutions and/or suspensions of the invention contain the lipopolysaccharides in admixture with excipients suitable for the manufacture thereof. Such excipients include a suspending agent, such as sodium carboxymethylcellulose, methylcellulose, hydroxypropylmethyl cellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadeaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservative such as ethyl of n-propyl p-hydroxybenzoate.

The pharmaceutical compositions of the invention are preferably in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

Formulations suitable for parenteral administration include aqueous and non-aqueous isotonic sterile injection solutions which may contain anti-oxidants, buffers, bacteriostats and solutes which render the formation isotonic with the blood of the intended recipient; and aqueous and non-aqueous sterile suspensions which may include suspending agents and thickening agents. The formulations may be presented in unit-dose or multi-dose sealed containers, for example, ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders of the kind previously described.

When using a lyophilized drug product, clinicians typically reconstitute the freeze-dried preparation in physiologically acceptable solutions. It is desirable to be able to store the reconstituted solution either at room temperature or under refrigeration. Freeze-dried preparations are rehydratable with water or an aqueous dextrose solution suitable for intravenous administration. Such reconstituted solutions can be stored at room temperature or refrigerated temperatures.

Each of the patents and publications cited herein are incorporated by reference herein in their entirety.

It will be apparent to one skilled in the art that various modifications can be made to the invention without departing from the spirit or scope of the appended claims.

What is claimed is:

1. A method for treating severe acute respiratory syndrome in a patient in need thereof comprising administering a therapeutically effective amount of a compound of Formula (1A) or a pharmaceutically acceptable salt thereof:

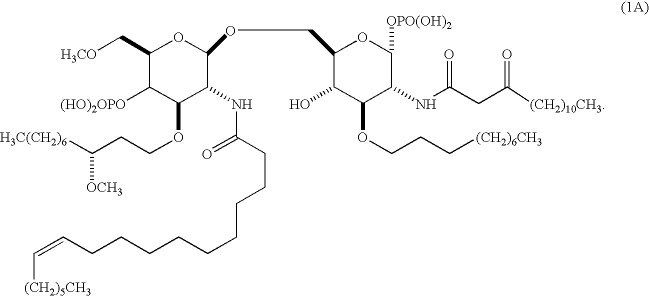

2. The method of claim 1, wherein the compound of Formula (1A) is in the form of a sodium salt.

3. The method of claim 1, wherein the compound of Formula (1A) or the pharmaceutically acceptable salt thereof is administered to the patient in an amount from 0.01 mg to 500 mg.

4. The method of claim 1, wherein the compound of Formula (1A) or the pharmaceutically acceptable salt thereof is administered to the patient in an amount from 1 mg to 50 mg.

5. The method of claim 1, wherein the compound of Formula (1A) or the pharmaceutically acceptable salt thereof is parenterally administered to the patient.

6. A method for treating severe acute respiratory syndrome in a patient in need thereof comprising administering a therapeutically effective amount of a compound of Formula (A) or a pharmaceutically acceptable salt thereof: 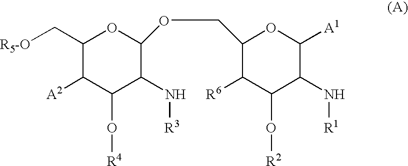
wherein R¹ is selected from the group consisting of: 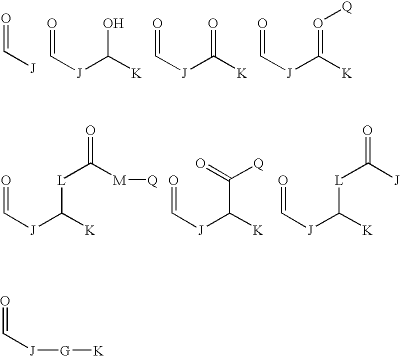
J, K and Q are each independently a straight or branched C₁₋₅ alkyl;
L is O, N or C;
M is Q or N;
G is N, O, S, SO or SO₂;
R² is a straight or branched C₅₋₁₅ alkyl;
R³ is selected from the group consisting of: 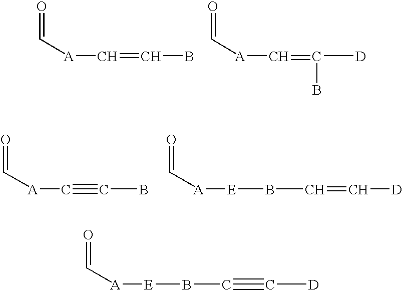
E is N, O, S, SO or SO₂;
A, B and D are each independently a straight or branched C₁₋₁₅ alkyl group;
R⁴ is a straight or branched C₄₋₂₀ alkyl group or 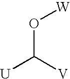
U and V are each independently a straight or branched C₂₋₁₅ alkyl group;
W is a hydrogen or a straight or branched C₁₋₅ alkyl group;
R⁵ is hydrogen, -J′, -J′-OH, -J′-O—K′, -J′-O—K′—OH or J′-O—PO(OH)₂;
J′ and K′ are each independently a straight or branched C₁₋₅ alkyl group;
R⁶ is hydroxy, halogen, a C₁₋₅ alkoxy group or a C₁₋₅ acyloxy group;
A¹ and A² are each independently selected from the group consisting of: 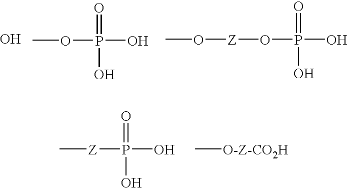
Z is a straight or branched C₁₋₁₀ alkyl group.

7. The method of claim 6, wherein the compound of Formula (A) or the pharmaceutically acceptable salt thereof is administered to the patient in an amount from 0.01 mg to 500 mg.

8. The method of claim 6, wherein the compound of Formula (A) or the pharmaceutically acceptable salt thereof is administered to the patient in an amount from 1 mg to 50 mg.

9. The method of claim 6, wherein the compound of Formula (A) or the pharmaceutically acceptable salt thereof is parenterally administered to the patient.

10. The method of claim 2, wherein the compound of Formula (A) is a compound of Formula (B) or a pharmaceutically acceptable salt thereof: 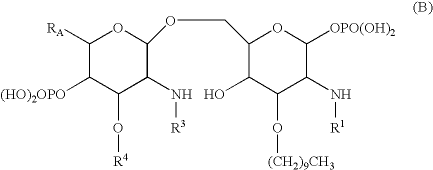
wherein R_{A}, R¹, R³ and R⁴ are as defined for the compound of Formula (A).

11. A method for treating sepsis caused by severe acute respiratory syndrome in a patient in need thereof comprising administering a therapeutically effective amount of a compound of Formula (A) or a pharmaceutically acceptable salt thereof: 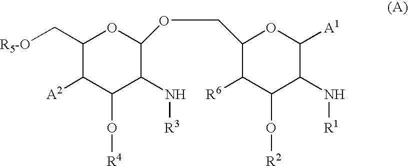
wherein R¹ is selected from the group consisting of: 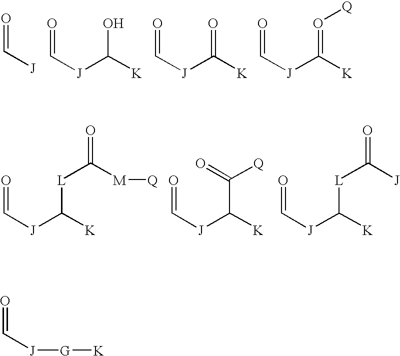
J, K and Q are each independently a straight or branched C₁₋₁₅ alkyl;
L is O, N or C;
M is O or N;
G is N, O, S, SO or SO₂;
R² is a straight or branched C₅₋₁₅ alkyl;
R³ is selected from the group consisting of: 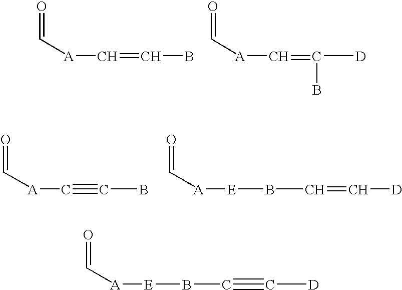
E is N, O, S, SO or SO₂;
A, B and D are each independently a straight or branched C₁₋₁₅ alkyl group;
R⁴ is a straight or branched C₄₋₂₀ alkyl group or 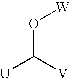
U and V are each independently a straight or branched C₂₋₁₅ alkyl group;
W is a hydrogen or a straight or branched C₁₋₅ alkyl group;
R⁵ is hydrogen, -J′, -J′-OH, -J′-O—K′, -J′-O—K′—OH or J′-O—PO(OH)₂;
J′ and K′ are each independently a straight or branched C₁₋₅ alkyl group;
R⁶ is hydroxy, halogen, a C₁₋₅ alkoxy group or a C₁₋₅ acyloxy group;
A¹ and A² are each independently selected from the group consisting of: 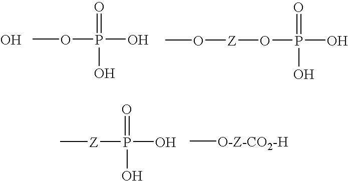
Z is a straight or branched C₁₋₁₀ alkyl group.

12. The method of claim 11, wherein the compound of Formula (A) or the pharmaceutically acceptable salt thereof is administered to the patient in an amount from 0.01 mg to 500 mg.

13. The method of claim 11, wherein the compound of Formula (A) or the pharmaceutically acceptable salt thereof is administered to the patient in an amount from 1 mg to 50 mg.

14. The method of claim 11, wherein the compound of Formula (A) or the pharmaceutically acceptable salt thereof is parenterally administered to the patient.

15. The method of claim 11, wherein the compound of Formula (A) is a compound of Formula (B) or a pharmaceutically acceptable salt thereof: 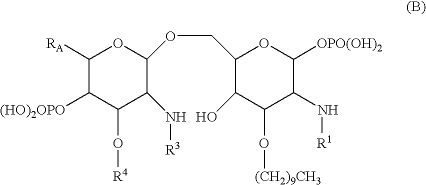
wherein R_{A}, R¹, R³ and R⁴ are as defined for the compound of Formula (A).

16. The method of claim 11, wherein the compound of Formula (A) is a compound of Formula (1) or a pharmaceutically acceptable salt thereof:

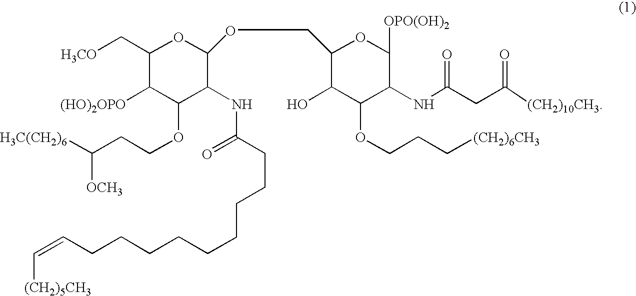

17. The method of claim 11, wherein the compound of Formula (A) is a compound of Formula (1A) or a pharmaceutically acceptable salt thereof:

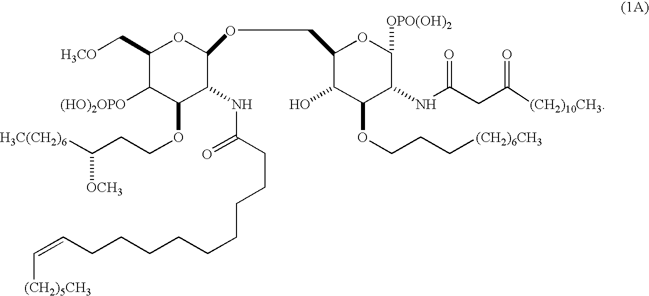

18. The method of claim 17, wherein the compound of Formula (1A) is in the form of a sodium salt.

19. The method of claim 17, wherein the compound of Formula (1A) or the pharmaceutically acceptable salt thereof is administered to the patient in an amount from 0.01 mg to 500 mg.

20. The method of claim 17, wherein the compound of Formula (1A) or the pharmaceutically acceptable salt thereof is parenterally administered to the patient.
